(19) United States
(12) Patent Application Publication
(10) Pub. No.: US 2025/0058296 A1
(43) Pub. Date: Feb. 20, 2025

(54) Precipitated silica for improved adsorption of volatile organic compounds

(71) Applicant: Alexander Lygin, Griesheim (DE)

(72) Inventor: Alexander Lygin, Griesheim (DE)

(21) Appl. No.: 18/762,747

(22) Filed: Jul. 3, 2024

(30) Foreign Application Priority Data
Aug. 14, 2023 (EP) 23020379

(51) Int. Cl. B01J 20/10; A23K 20/105; A61K 31/21; A61K 47/02; B01J 20/28; B01J 20/30

(52) U.S. Cl. CPC B01J 20/103; A23K 20/105; A61K 31/21; A61K 47/02; B01J 20/28004; B01J 20/28011; B01J 20/28059; B01J 20/28061; B01J 20/28064; B01J 20/3078

(57) ABSTRACT

Dry mixture comprising: (a) from 10% to 99% by weight of a precipitated silica having: (a1) BET surface area of from 30 m²/g to 700 m²/g, (a1) numerical median particle size d₅₀ determined by laser diffraction method of from 100 μm to 600 μm, (a3) silanol group density d_{SiOH} determined by reaction of the pre-dried silica with lithium aluminum hydride of from 1.55 to 3.65 SiOH/nm² and (b) from 1 wt % to 90 wt % of at least one organic compound: (b1) selected from selected from an alcohol, a diol, an aldehyde, a ketone, a carboxylic acid, a carboxylic esters, a nitrate ester, an amine, and/or oxygen and/or nitrogen heterocycle, (b2) having 2 to 12 carbon atoms, (b3) having a vapour pressure of at least 0.01 kPa at 293 K, wherein the organic compound is adsorbed on the precipitated silica, the process for manufacturing thereof.

### FIELD OF THE INVENTION

Present invention relates to a dry mixture comprising precipitated silica and a volatile organic compound adsorbed thereon, a process for preparation such a mixture and the use thereof, especially as a food additive or animal feed additive.

Description of the prior art and the technical problem Various organic compounds are often adsorbed on carrier materials based on precipitated silica, the resulting adsorbate/carrier systems being used e.g. as food additives or animal feed additives.

Thus, EP1241136A2 discloses preparation of precipitated silicas for use as carrier materials e.g. for choline chloride. One key feature of such silicas for use as carrier materials is their adsorption capacity, which is often related to high BET surface area and porosity. Many development efforts have therefore been directed to maximizing this adsorption capacity of precipitated silica carriers.

Another general problem with silica carriers waiting for a solution occurs during the handling of adsorbate/carrier systems and is associated with the use of volatile organic compounds (VOCs). Such compounds may easily be lost via evaporation prior to their intended use, e.g. during storage, transport, and other handling steps. This decreases the content of the useful organic component in the system and may lead to environmental pollution by VOC emissions, unpleasant smell, and many other negative consequences.

WO2018149756A1 discloses powdery formulations comprising nitrate esters such as 3-nitrooxypropanol (3-NOP) adsorbed on a silica carrier with a particle size D(v, 0.5) of ≤320 μm, e.g. a precipitated silica. It is emphasized in this patent application, that evaporation of 3-NOP during the storage is a challenge. According to the patent application, variation of particle size of the silica carrier may solve this problem to some extent. However, the examples of WO2018149756A1 show that 3-NOP retention was still incomplete, even with the optimized carriers. Hence, the problem of retention of volatile adsorbates on a silica carrier still existed in this case.

Surprisingly, it has now been found that precipitated silica carriers with specific reduced silanol group density may be efficiently used for solving the afore-mentioned problem, i.e. reducing the desorption of volatile compounds from precipitated silica, if used as carriers. The required reduced silanol group density may be achieved e.g. via thermal treatment under particular reaction conditions. It turned out that there is an optimum of absorption capacity of a precipitated silica carrier corresponding to a specific narrow range of silanol group density.

Mentioned above EP1241136A2 shows preparation of several precipitated silicas including their granulation by spray drying of aqueous suspensions. According to the experimental data, silanol group density measured with Sears method, i.e. alkaline titration, does not actually correlate with water content of silica samples. Thus, the samples with the same water content (examples 4 and 9, 6 wt % water) show very different silanol group densities (29.5 vs. 18.4), whereas the samples with similar Sears values (examples 1 and 2, Sears 31.3 vs. 30.9) show very different water contents (6.2 vs. 5.3 wt %). The data (example 9) also suggests that silicas with the lowest silanol group density would lead to the lowest adsorption capacity as confirmed by the DBP adsorption values. This drives the skilled person to the conclusion not to reduce the silanol group density if a better adsorption is desired.

U.S. Pat. No. 5,395,605 discloses preparation of precipitated silicas by heat treatment at the temperature of at least 700° C. for at least one minute. Such silicas have “lower water uptake” and are proposed for use as reinforcing fillers with a good dispersibility, for e.g. silicone formulations. U.S. Pat. No. 5,395,605 does not suggest using thermally treated silicas as adsorbents.

US20150209252A1 discloses toothpaste compositions, i.e. not free-flowing “dry mixtures”, but viscose, pasty liquid compositions, containing typically about 20 wt % of heat-treated in the temperature range 150° C.-1000° C. precipitated silicas. These heat-treated silicas show reduced water content and “lower affinity for water” compared to non-heat-treated silicas and are used primarily as abrasive materials.

Similarly, WO2022101015A1 describes precipitated silicas with a number of silanol groups (determined by mass loss in the temperature rage 200° C.-800° C.) not higher than 2.00 OH/nm² and CTAB of 5-45 m²/g prepared by thermal treatment of corresponding “starting silicas” with CTAB of less than 50 m²/g at 400-650° C. for 1 to 180 min. Such thermally treated silicas are reported to allow less decomposition of hydrogen peroxide in the corresponding toothpaste formulations.

US20160159654A1 discloses producing calcined in the range 600° C.-1200° C. precipitated silicas having “low hygroscopicity” and high dispersibility in resin.

Technical problem solved by the present invention is that of providing an adsorbate/carrier system based on precipitated silica carrier and containing volatile organic compounds as an adsorbate, wherein the volatile compound shows strong adsorption and reduced desorption/evaporation rate from the carrier. Additionally, the carrier should be characterized by overall high adsorption capacity and be able to adsorb relatively large quantities of the adsorbate and form free-flowing powdery or granular adsorbate/carrier mixtures. Additionally, high mechanical stability, low attrition and fines formation is desired as well as low extent of aggregation of the carrier, which would improve handling of such systems.

### Solution

The present invention provides dry mixture comprising:
(a) from 10% to 99% by weight of a precipitated silica having:
   (a1) BET surface area of from 30 m²/g to 700 m²/g,
   (a1) numerical median particle size d₅₀ determined by laser diffraction method of from 100 μm to 600 μm,
   (a3) silanol group density d_{SiOH} determined by reaction of the pre-dried silica with lithium aluminum hydride of from 1.55 to 3.65 SiOH/nm²
and
(b) from 1 wt % to 90 wt % of at least one organic compound:
   (b1) selected from selected from an alcohol, a diol, an aldehyde, a ketone, a carboxylic acid, a carboxylic esters, a nitrate ester, an amine, and/or oxygen or nitrogen heterocycle,
   (b2) having 2 to 12 carbon atoms,
   (b3) having a vapour pressure of at least 0.01 kPa at 293.15 K, wherein the organic compound is adsorbed on the precipitated silica.

As discussed above, based on the known prior art, the person skilled in the art trying to optimize the adsorption/desorption of polar volatile compounds on precipitated silica carriers would arrive at the conclusion that precipitated silicas with reduced silanol group density, e.g. those prepared by thermal treatment, show lower affinity for and adsorption of polar volatile compounds such as water. This teaches the skilled person away from the idea of using adsorbate/carrier systems with reduced silanol group density of silica carrier.

### Precipitated Silica

The term “dry mixture” means in the context of the present invention powdery or granular free-flowing mixture. Such “dry mixture” typically contains a powdery or granular porous carrier material and an adsorbate adsorbed on the carrier. The terms “adsorbate” and “adsorbed” are intended to mean both adsorption of the organic compound on the silica surface as well as absorption thereof in the pores of the silica. The adsorbate, typically in a liquid form, is normally essentially completely and relatively firmly attached to the surface of the carrier's pores so that essentially no non-adsorbed liquid adsorbate can be found in the space between carrier particles. Thus, “dry mixture” of the present invention differs from any liquid or pasty composition comprising a carrier and a liquid adsorbate in that they are free-flowing and typically contain relatively high content of porous carrier.

The dry mixture according to the invention comprises from 10% to 99%, preferably 20% to 98%, more preferably 25% to 97%, more preferably 30% to 96%, more preferably 40% to 95%, more preferably 50% to 95% by weight of precipitated silica.

The term “precipitated silica” referred to in the present invention is well known in the art. Precipitated silicas are typically produced by the reaction of an alkaline silicate solution, e.g., sodium silicate, with a mineral acid. Commercially, sulfuric acid is mainly used, although other acids such as hydrochloric acid or carbonic acid (carbon dioxide) can be applied as well. The acid and the sodium silicate solution are usually added simultaneously with agitation to water. Precipitated silica arises when silica is precipitated from this dispersion by the neutralization reaction and the generation of by-product sodium salt (sodium sulfate). The precipitated silica consists of aggregates (secondary particles) of primary (or ultimate) colloidal silica particles. The primary particles are mostly spherical and usually have a diameter in the range between 5 nm and 50 nm. The primary particles in the aggregates are covalently bonded to one another by the formation of siloxane bonds. The aggregates are three-dimensional clusters of these primary particles. The aggregates usually have diameters of up to 500 nm. The aggregates are typically linked into a massive gel network during the preparation process. The aggregates themselves can be physically linked to larger agglomerates of up to 100 μm in diameter by the formation of hydrogen bonds between the silanol groups on their surfaces before milling). The median agglomerate size is typically about 20 μm-50 μm in diameter (before milling). Precipitated silicas may be treated in many known ways, e.g. milled, subjected to hydrophobization etc.

The precipitated silica in the invention can have a BET surface area of 30 m²/g to 700 m²/g, preferably of 50 m²/g to 600 m²/g, more preferably of 100 m²/g to 500 m²/g, more preferably of 150 m²/g to 450 m²/g, most preferably of 150 m²/g to 250 m²/g. The specific surface area, also referred to simply as BET surface area, can be determined according to DIN 9277:2014 by nitrogen adsorption in accordance with the Brunauer-Emmett-Teller method. BET surface area of the precipitated silica in the above-specified preferred range, particularly between 150 m²/g and 450 m²/g, turned out to be optimal in terms of maximal adsorption of the organic compound per mass of the silica at acceptable mechanical strength leading to less mechanical attrition and fines formation and better handling.

The silanol group density d_{SiOH} is the average number of silanol groups relative to BET surface area of the silica powder (in nm), expressed in SiOH/nm². This parameter d_{SiOH} can be determined by the method described in detail on page 8, line 17 thru page 9, line 12 of EP 0725037 A1 by reaction of the silica powder (or granules) with lithium aluminium hydride. Silica is usually pre-dried under vacuum (10⁻² mbar) for 30 minutes at 130° C. prior to the measurement. The silanol (SiOH) groups of the silica are reacted with lithium aluminium hydride (LiAIH₄), the quantity of gaseous hydrogen formed during this reaction and thus the amount of silanol groups in the sample n_{SiOH} (in mmol SiOH/g) is determined. Using the corresponding BET surface area (in m²/g) of the tested material, the silanol group content in mmol SiOH/g can be easily converted in number d_{SiOH} of silanol groups relative to BET surface area:

d_{OH}[SiOH/nm²]=(n_{SiOH}[mmol SiOH/g]×N_{A})/(BET[m²/g]×10²¹),

wherein N_{A} is Avogadro number (˜6.022*10²³).

The precipitated silica in the present invention has a silanol group density d_{SiOH} of from 1.55 SiOH/nm² to 3.65 SiOH/nm², preferably from 1.60 SiOH/nm² to 3.65 SiOH/nm², more preferably from 1.70 SiOH/nm² to 3.65 SiOH/nm², more preferably from 1.80 SiOH/nm² to 3.65 SiOH/nm², more preferably from 2.00 SiOH/nm² to 3.60 SiOH/nm², more preferably from 2.20 SiOH/nm² to 3.55 SiOH/nm², more preferably from 2.40 SiOH/nm² to 3.50 SiOH/nm², more preferably from 2.60 SiOH/nm² to 3.45 SiOH/nm², more preferably from 2.70 SiOH/nm² to 3.40 SiOH/nm². Silanol group densities in the above-mentioned narrow range, particularly 2.00 SiOH/nm² to 3.60 SiOH/nm² were found to be particularly suitable for the optimal retention of volatile organic compounds adsorbed on the surface of silica carrier. On the other hand, interestingly, precipitated silicas with silanol group densities in a narrow range from 3.35 SiOH/nm² to 3.65 SiOH/nm², particularly 3.40 SiOH/nm² to 3.60 SiOH/nm² were found to show improved retention of volatile compounds while simultaneously having no or only the smallest extent of agglomeration of silica particles, which is a useful feature while handling such systems.

The silica used in the inventive mixture is preferably characterized by a di-(2-ethylhexyl) adipate (DOA) absorption capacity of at least 150 mL DOA/100 g silica, more preferably at least 150 mL DOA/100 g silica. The measurement of DOA absorption capacity of the silica may be done following ISO19246:2016. Thus, preferred precipitated silicas used in the present invention are characterized by relatively high adsorption values of typical organic adsorbates, which differs them from silicas having low BET surface area and/or porosity and/or pore diameters in sub-optimal for adsorption range, i.e. too small or too large pore diameters.

The precipitated silica in the present invention is preferably hydrophilic, i.e. not subjected to hydrophobization by any kind of hydrophobizing surface treatment. As such, the precipitated silica in the invention usually has a relatively low carbon content of less than 3% by weight, preferably less than 1% by weight, more preferably less than 0.5% by weight, more preferably less than 0.1% by weight. The carbon content can be determined by elemental analysis according to EN IS03262-20:2000 (Chapter 8). The analysed sample is weighed into a ceramic crucible, provided with combustion additives, and heated in an induction furnace under an oxygen flow. The carbon present is oxidized to CO₂. The amount of CO₂ gas is quantified by infrared detectors. The stated carbon content of silica powder according to the invention refers to all carbon-containing components of the silica except for non-combustible compounds such as e.g. silicon carbide.

The precipitated silica used in the present invention may be in the form of coarse powder, or granules, i.e. larger, aggregated particles.

The precipitated silica in the present invention has a numerical median particle size d₅₀ of from 100 μm to 1000 μm, more preferably from 100 μm to 800 μm, more preferably from 200 μm to 600 μm, more preferably from 250 μm to 550 μm, more preferably from 300 μm to 500 μm. A numerical median particle size of the silica can be determined according to ISO 13320:2009 by laser diffraction particle size analysis. The resulting measured particle size distribution is used to define the median d₅₀, which reflects the particle size not exceeded by 50% of all particles, as the numerical median particle size. Median particle size of the precipitated silica in the above-specified preferred range of 100 μm to 1000 μm, particularly between 200 μm and 600 μm, turned out to be optimal in terms of handling of the resulting adsorbate/carrier system (inventive mixture), i.e. minimal attrition and fines formation, loading and other aspects. Surprisingly, the major technical effect of the invention, i.e. the improved retention of volatile organic compound in the adsorbate/carrier system turned out to be particularly pronounced for such granular systems as opposed to powdery mixtures.

Tamped densities (also referred to as “tapped density”) of various pulverulent or coarse-grain granular materials can be determined according to DIN ISO 787-11:1995 “General methods of test for pigments and extenders—Part 11: Determination of tamped volume and apparent density after tamping”. This involves measuring the apparent density of a bed after agitation and tamping. The precipitated silica in the invention preferably has a tamped density of more than 30 g/L, more preferably of 30 g/L to 600 g/L, more preferably of 50 g/L to 500 g/L, more preferably of 70 g/L to 450 g/L, more preferably of 150 g/L to 450 g/L, more preferably of 200 g/L to 400 g/L. Tamped density of the precipitated silica in the above-specified range, particularly between 150 g/L and 450 g/L, turned out to be optimal in terms of handling of the resulting adsorbate/carrier system, i.e. minimal attrition and fines formation, loading and other aspects.

Loss on drying (LOD) of the precipitated silica in the present invention is preferably less than 5.0 wt %, more preferably less than 4.5 wt %, more preferably less than 4.0 wt %, more preferably less than 3.5 wt %, more preferably less than 3.0 wt %, more preferably less than 2.5 wt %. Loss on drying can be determined after 2 hours at 105° C. following ISO 787-2.

LOD value only reflects the partial water content of the silica, as it shows the water being dried off at 105° C. The total water content of the precipitated silica may be measured by the Karl-Fischer titration method using any suitable Karl-Fischer titrator according to STN ISO 760. Water content of the precipitated silica in the invention determined by Karl-Fischer method is preferably lower than 6.0% by weight, more preferably from 0.5% by weight to 5.5% by weight, more preferably from 0.7% by weight to 5.2% by weight, more preferably from 0.9% by weight to 5.1% by weight, more preferably from 1.0% by weight to 5.0% by weight, more preferably from 1.5% by weight to 4.8% by weight, more preferably from 2.0% by weight to 4.5% by weight.

Water content of precipitated silicas may depend on their BET surface area, typically being higher for higher surface areas. Therefore, the ratio of water content to BET surface area, which is independent on BET surface area, sems to be more suitable for characterization of the thermal treated precipitated silicas in the present invention. The ratio of water content of the silica in % by weight, as determined by Karl-Fisher titration to BET surface area of the silica in m²/g is preferably from 0.005 wt %×g/m² to 0.030 wt %×g/m², more preferably from 0.006 wt %×g/m² to 0.028 wt %×g/m², more preferably from 0.008 wt %×g/m² to 0.025 wt %×g/m², more preferably from 0.010 wt %×g/m² to 0.023 wt %×g/m². This above-specified value of water content—to BET ratio is characteristic for precipitated silicas in the inventive mixture having relatively low water content at simultaneously relatively high BET surface area. This set of features is preferred for having both high adsorption and retention of the adsorbed organic compound.

The porosity of precipitated silica particles typically depends on the size of the silica primary particles and the kind of their aggregation and agglomeration. The pores are formed by the spaces between the primary particles, and the aggregates.

The precipitated silica in the inventive mixture is preferably characterized by a total volume of mesopores, i.e. the pores in the diameter range of 2 nm to 50 nm, of 0.65 mL/g to 0.90 mL/g, as determined by the mercury intrusion method according to DIN ISO 15901-1. The principle of this method firstly described by H. L Ritter and L. C Drake in Ind. Eng. Chem. Anal. Ed. 17 (1945) pp. 782-786 and pp 787-791, is based on measurement of the volume of mercury pressed into a porous solid body as a function of the pressure applied.

The average pore diameter of the pores of the precipitated silica is typically in the range from 2 nm to 40 nm, preferably 3 nm to 30 nm, more preferably 4 nm to 20 nm, as determined by nitrogen adsorption method.

Precipitated silica in the invention preferably has a pH of 2.5 to 7.0, more preferably 3.5 to 6.5, more preferably 3.8 to 6.2, more preferably 4.0 to 6.0, more preferably 4.5 to 5.5. pH value of the silica can be measured in a 5 wt % silica dispersion in water according to ISO 787-9. It turned out that the maximal adsorption and retention of the organic compound is observed in the above-mentioned preferred range of pH, particularly at pH from 4.0 to 6.0.

### Organic Compound

The dry mixture of the invention comprises 1% to 90%, preferably 2% to 50%, more preferably 3% to 40%, more preferably 4% to 30%, more preferably 5% to 20% by weight of at least one organic compound adsorbed on the precipitated silica.

The term “adsorbed” is used in the present invention in its typical, well-known for the person skilled in the art meaning referred to the result of adsorption, i.e. the process in which atoms, ions or molecules of a substance (in the present invention: organic compound) adhere to a surface of the adsorbent.

The term “organic compound” in the context of the present invention is used in its typical, well-known for the person skilled in the art meaning and refers to compounds in which one or more atoms of carbon are covalently linked to atoms of other elements, most commonly hydrogen, oxygen, or nitrogen.

The present invention relates especially to the so-called “volatile” organic compounds adsorbed on the silica carrier. The term “volatile organic compound (VOC)” is used in the context of the present invention in its common, well-known for the skilled person meaning. The VOC-directive [Official Journal of the European Communities; Brussels: 1999. EU VOC-directive: Council Directive 1999/13/EC of 11 Mar. 1999 on the limitation of emissions of volatile organic compounds due to the use of organic solvents in certain activities and installations.] defines a volatile organic compound (VOC) as an organic compound having at 293.15 K (=20° C.) a vapour pressure of 0.01 kPa (=0.075 mmHg) or more.

The organic compound in the present invention can have a boiling point of up to 300° C., preferably 20° C.-280° C., more preferably 30° C.-260° C., more preferably 50° C.-250° C., more preferably 100° C.-220° C., at a pressure of 1 atm. The organic compound is preferably in a liquid form. All pressures referred to in the present invention are absolute pressures.

Volatile organic compounds usually have relatively low boiling points. However, sometimes the compounds having relatively high boiling points may have quite high vapor pressure at 293.15 K (20° C.) and are therefore volatile.

The present invention relates to relatively volatile compounds, whose retention in adsorbed form on the precipitated silica of the dry mixture according to the invention is improved by the invention. Thus, camphor having a boiling point of 209° C., is reported to have vapor pressure of about 24 Pa at 293.15 K and is therefore volatile according to the definition given above, while naphthalene having a boiling point of 218° C. and a vapor pressure of about 7 Pa at 293.15 Pa is not volatile according to the definition above.

The organic compound has 2 to 12, more preferably 2 to 10, more preferably 3 to 10, more preferably 3 to 8 carbon atoms in its structure.

The organic compound is selected from an alcohol, a diol, an aldehyde, a ketone, a carboxylic acid, a carboxylic ester, a nitrate ester, an amine, and/or oxygen or nitrogen heterocycle.

The organic compound can have unsaturated and/or saturated linear, branched, cyclic and/or aromatic structure units.

A non-exhaustive list of suitable alcohols includes linear, branched or cyclic propanol, butanol, pentanol, hexanol, heptanol, octanol, nonanol. Decanol, as well as estragole, ethyl maltol, terpineol, eucalyptol, linalool, dihydromyrcenol.

Some examples of suitable aldehydes include benzaldehyde; of ketones: camphor; of carboxylic acids: propionic acid, butyric acid, pentanoic acid, hexanoic acid; of carboxylic esters: benzyl acetate, methyl butyrate, ethyl butyrate, hexyl acetate, ethyl acetate, isoamyl acetate, methyl salicylate; of nitrate esters: 3-nitrooxypropanol; of amines: primary, secondary or tertiary amines such as propylamine, butylamine; of oxygen and/or nitrogen heterocycles: furan, pyrrole.

In one preferred embodiment of the invention, the organic compound is an alcohol and/or a nitrate ester with 3 to 10 carbon atoms. Thus, in one particularly preferred embodiment, the organic compound is 3-nitrooxypropanol (3-NOP).

In another preferred embodiment of the invention, the organic compound is a mixture of ethylene glycol and 3-nitrooxypropanol.

In another preferred embodiment of the invention, the organic compound is a carboxylic acid with 3 to 10 carbon atoms.

In another preferred embodiment of the invention, the organic compound is an aldehyde, a ketone, a carboxylic ester and/or an amine with 3 to 10 carbon atoms.

In a preferred embodiment of the invention, the dry mixture comprises a compound of formula (I) as an organic compound

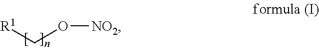

wherein
n is an integer from 1 to 10, more preferably from 2 to 8,
R¹ is selected from the group consisting of H, C₁-C₆ alkyl, phenyl, —OH, —NH₂, —CN, —COOH, —O(C═O)R², and —ONO₂, and
R² is C₁-C₆ alkyl, phenyl, pyridyl such as preferably 2-pyridyl with the proviso that when n is >3 the hydrocarbon chain may be interrupted by —O— or —NH—.

### Process for Producing the Dry Mixture

The invention further provides a process for manufacturing the inventive dry mixture, comprising:
step (a) of thermal treatment of a precipitated silica having a BET surface area of from 30 m²/g to 700 m²/g, a numerical median particle size d₅₀ determined by laser diffraction method of from 100 μm to 1000 μm and a silanol group density d_{SiOH} determined by reaction of the pre-dried silica with lithium aluminum hydride of more than 3.65 SiOH/nm² at a temperature of from 120° C. to 700° C., for at least 1 minute and
step (b) of adding at least one organic compound selected from selected from an alcohol, a diol, an aldehyde, a ketone, a carboxylic acid, a carboxylic esters, a nitrate ester, an amine, and/or oxygen or nitrogen heterocycle, having 2 to 12 carbon atoms and having a vapour pressure of at least 0.01 kPa at 293.15 K to the thermally treated silica prepared in step (a) and adsorbing the organic compound on the silica.

The process according to the present invention combines thermal treatment of selected precipitated silica under specified conditions (step (a)) and adsorption of selected volatile organic compounds on the pre-treated silica carrier (step (b)).

The precipitated silica used in step (a) of the inventive process is referred to as “starting precipitated silica” in the context of the present invention.

The starting precipitated silica in the invention can have a BET surface area of 30 m²/g to 700 m²/g, preferably of 50 m²/g to 600 m²/g, more preferably of 100 m²/g to 500 m²/g, more preferably of 150 m²/g to 450 m²/g, most preferably of 150 m²/g to 250 m²/g, as determined according to DIN 9277:2014 by nitrogen adsorption in accordance with the Brunauer-Emmett-Teller method.

The starting precipitated silica in the present invention has a silanol group density d_{SiOH} of more than 3.65 SiOH/nm², preferably from 3.65 SiOH/nm² to 8.00 SiOH/nm², more preferably from 3.70 SiOH/nm² to 6.00 SiOH/nm², as determined by the method described in detail on page 8, line 17 thru page 9, line 12 of EP 0725037 A1 by reaction of the silica powder (or granules) with lithium aluminium hydride.

The starting precipitated silica in the present invention preferably has a numerical median particle size d₅₀ of from 100 μm to 1000 μm, more preferably from 200 μm to 600 μm, more preferably from 250 μm to 550 μm, more preferably from 300 μm to 500 μm, as determined according to DIN ISO 787-11:1995.

It is believed that thermal treatment in step a) of the inventive process leads to reducing the silanol group density by condensation of such groups and formation of O—Si—O bridges.

It has been found that the extent of the required silanol group density reduction of the precipitated silica in step (a) of the inventive process depends on the combination of (1) the temperature of thermal treatment and (2) duration of thermal treatment.

Step (a) is carried out at 120° C. to 700° C., preferably at 120° C. to 600° C., more preferably at 120° C. to 500° C., more preferably at 120° C. to 450° C., more preferably at 150° C. to 450° C., more preferably at 200° C. to 400° C.

The duration of step (a) is at least 1 minute, preferably at least 5 minutes, more preferably at least 10 minutes, more preferably at least 30 minutes, more preferably at least 1 hour.

Step (a) of the inventive process is preferably carried out at a temperature of from 120° C. to 450° C. for at least 10 minutes.

Importantly, the same extent of the silanol group density reduction may be achieved using the higher temperature and the shorter duration of the thermal treatment or using the lower temperature and the longer duration of the process. Thus, process conditions may be adjusted within the specified temperature and duration ranges so that the required silanol group density of the precipitated silica can be achieved.

Thermal treatment in step (a) of the inventive process can be carried out under a gas atmosphere of typically at least 10 mbar, preferably at least 100 mbar, more preferably at least 500 mbar, more preferably 0.5 bar to 2.0 bar, more preferably 0.8 bar to 1.2 bar. Thus, both reduced pressure (<1 bar) and overpressure compared to atmospheric pressure may be used in the inventive process.

Any suitable gas, preferably the one not reacting with precipitated silica under process conditions, may constitute such gas atmosphere. Dried air and/or nitrogen are preferably used as gases in the process.

Step (a) of the inventive process may be carried out batchwise under the above-mentioned suitable gases or continuously, in the latter case preferably under continuous flow of such suitable gases. The preferred ratio of the employed total gas volume (under standard conditions) to the employed total mass of the precipitated silica is in the range 0.1 m³/kg to 10 m³/kg, more preferably 0.3 m³/kg to 6 m³/kg, more preferably 0.5 m³/kg to 3 m³/kg. In a continuous process, the above-mentioned ratio of the employed total gas volume (under standard conditions) to the employed total mass of the precipitated silica corresponds to the ratio of gas flow (in Nm³/h) to silica mass flow (kg/h).

Essentially no water except for the adsorbed water contained in the (starting) precipitated silica prior to carrying out step (a) of the process, is preferably added before, during or after carrying out step a) of the inventive process. Small amounts of water may be introduced into the process during thermal treatment in step (a) e.g. as humidity with the used carrier gas. However, larger amounts of water, e.g. treatment silica with steam or saturated with water air, are not recommended. “Essentially no water” means in the context of the present invention that the total amount of water introduced before, during or after carrying out step (a) does not exceed 50%, preferably does not exceed 20%, more preferably does not exceed 10% of the total amount of water removed from the starting precipitated silica during the thermal treatment process.

In this way, the undesired adsorption of water on the precipitated silica can be avoided in the process and the required reduction of the silanol group density may be achieved at relatively low process temperatures, which in turn allows keeping the BET surface area and the adsorption capacity of the silica on the high level.

Gas atmosphere during the thermal treatment process step (a) preferably has maximal water content of 0.5 g water/L gas, preferably maximally 0.3 g water/L gas, more preferably maximally 0.1 g water/L gas, more preferably maximally 0.05 g water/L gas.

The thermal treatment in step (a) is preferably carried out under movement of the silica, at least temporally during the thermal treatment step a), at the motion rate of at least 1 cm/min, more preferably at least 5 cm/min, more preferably at least 25 cm/min, more preferably at least 50 cm/min. It has been found that such a movement of silica during the thermal treatment process leads to much less aggregation, better temperature control and more homogeneous product properties.

The inventive process can be carried out in any suitable apparatus allowing keeping the precipitated silica particles at the above-specified temperature for a specified period of time, while moving the silica. Some suitable apparatuses are fluidized bed reactors and rotary kilns. Rotary kilns, particularly those with a diameter of 1 cm to 2 m, preferably 5 cm to 1 m, more preferably 10 cm to 50 cm, are preferably used in the inventive process. The silica can be moved at least temporally during the thermal treatment step a), but preferably, the silica is being moved at the specified motion rate continuously for the whole duration of the thermal treatment step (a) of the inventive process. The motion rate in a rotary kiln corresponds to circumferential speed of this reactor type. The motion rate in a fluidized bed reactor corresponds to the carrier gas flow rate (fluidization velocity).

Precipitated silica obtained in step (a) of the process can be cooled down to 15° C.-200° C., more preferably 20° C.-50° C., prior to carrying out step (b) of the process. This cooling step is preferably carried out under exclusion of water, e.g. in a flow of dried gas such as air or nitrogen.

In step b) of the inventive process, the precipitated silica subjected to thermal treatment in step a) and optionally cooled down to 15° C.-200° C., preferably 20° C.-50° C., is subjected to adding at least one organic compound, comprising at least one oxygen (O) atom and/or at least one nitrogen (N) atom, and having a vapour pressure of at least 0.01 kPa at 293.15 K and adsorbing of the organic compound on the silica.

Step (b) of the process preferably directly follows step (a) and optional cooling the silica, i.e. no longer transport and/or storage of silica between steps (a) and (b) of the process takes place. In any case, silica thermally treated in step (a) of the process is preferably kept under essential exclusion of water until adsorption of the organic compound in step (b) takes place. In this case, the reduced silanol group density of the thermally-treated precipitated silica can be efficiently kept until the adsorption step.

Step b) of the process according to the invention can be carried out at a temperature of 10° C. to 250° C., preferably 20° C. to 50° C. for 1 second to 24 hours, preferably 1 minute to 1 hour.

The organic compound may be added in any suitable way, e.g. by spraying ensuring the homogeneous distribution thereof on the silica surface.

Step (b) can be conducted under exclusion of water and/or under protective gas, such as, for example, nitrogen. This step can be carried out in any suitable apparatus such as mixer equipped with a spraying means, either continuously or batchwise.

### Use of the Mixture

The inventive mixture can be used as food additive and/or animal feed additive, for transport and/or storage of chemicals and chemical intermediates, as detergent, additive for washing machines, as paint or coating additive, as component of rubber or sealant, e.g. silicone compositions, as perfuming or flavoring ingredient, and in toothpaste.

Preferably, the mixture according to the invention can be used as such or as a component of animal feed products or in the production of feed products.

Particularly preferably, the inventive mixture comprising the compound of formula (I) described above such as 3-nitrooxypropanol (3-NOP) is used as a component of animal feed products, especially for reducing methane production by ruminants (e.g. cattle, sheep, goats).

Equally preferably, the inventive mixture comprising carboxylic acids as organic compound can be used as a component of animal feed products, especially for providing antimicrobial effect.

Especially in the case if the mixture according to the present invention is used a component of a animal feed product, it can additionally be coated with customary in the art coatings such as wax or fats. If present, such coating is generally applied in amounts of 2 to 50 wt.-% based on the total weight of the mixture. Advantageously, the coating comprises at least one wax and/or at least one fat, which has a dropping point of from 30 to 85° C. The dropping point of a material as used herein refers to the temperature (in ° C.) when the material begins to melt under standardized conditions. Thus, the material is heated so long until it changes the state of matter from solid to liquid. The dropping point is the temperature when the first dropping is released from the material. The determination of the dropping point (“Tropfpunkt”) is carried out as described in the standard norm DIN ISO 2176.

Particularly suitable waxes to be used as coating in the context of the present invention include organic compounds consisting of long alkyl chains, natural waxes (plant, animal) which are typically esters of fatty acids and long chain alcohols as well as synthetic waxes, which are long-chain hydrocarbons lacking functional groups.

Particularly suitable fats to be used as coating in the context of the present invention include a wide group of compounds which are soluble in organic solvents and largely insoluble in water such as hydrogenated fats (or saturated fats) which are generally triesters of glycerol and fatty acids. Suitable fats can have natural or synthetic origin. It is possible to hydrogenate a (poly)unsaturated fat to obtain a hydrogenated (saturated) fat.

Preferred examples of waxes and fats to be used as coating according to the present invention are glycerine monostearate, carnauba wax, candelilla wax, sugarcane wax, palmitic acid, stearic acid hydrogenated cottonseed oil, hydrogenated palm oil and hydrogenated rapeseed oil as well as mixtures thereof.

Another preferred use of the inventive dry mixture is that as a perfuming or flavouring ingredient, e.g. in the corresponding compositions. A perfuming or flavouring ingredient, within the framework of the invention, designates a compound typically used in perfumery or flavour industry able to impart an odour, or to improve, enhance or modify the odour or taste properties of the composition to which it is added. Usually, these ingredients will form a complex mixture of volatile ingredients of natural or synthetic origin. The nature of these ingredients can be found in specialised books, e.g. in S. Arctander (Perfume and Flavor Chemicals, Montclair N.J., USA 1969), or in Fenaroli's Handbook of Flavour Ingredients, CRC Press or yet in Synthetic Food Adjuncts by M. B. Jacobs, van Nostrand Co., Inc. or similar textbooks of reference. Typical perfuming or flavouring ingredients are typically used in liquid form.

### EXAMPLES

### Analytical Methods

The number of silanol groups relative to BET surface area d_{SiOH} [in SiOH/nm²] was determined by reaction of the pre-dried samples of silica with lithium aluminium hydride solution as described in detail on page 8, line 17 thru page 9, line 12 of EP 0725037 A1.

Specific BET surface area [in m²/g] was determined according to DIN 9277:2014 by nitrogen adsorption in accordance with the Brunauer-Emmett-Teller method.

Water content [in wt. %] was determined by Karl Fischer titration using a Karl Fischer titrator.

Tamped density [in g/L] was determined according to DIN ISO 787-11:1995 “General methods of test for pigments and extenders—Part 11: Determination of tamped volume and apparent density after tamping”.

Oil absorption of precipitated silica (DOA absorption, in mL/100 g) was determined using di-(2-ethylhexyl) adipate (DOA) according to ISO 19246:2016.

Pore volume [mL/g] for mesopores (2-50 nm) was determined by nitrogen adsorption method according to DIN 66134.

### Silica Samples

Silica used in Comparative Example 1 was precipitated silica Sipernat® 2200 (manufacturer: Evonik Operations GmbH).

### Preparation of Silica Samples for Examples 2-6 and Comparative Example 7

Precipitated silica Sipernat® 2200 (manufacturer: Evonik Operations GmbH) was subjected to thermal treatment in a rotary kiln of ca. 160 mm diameter and 2 m length at a temperature from 120° C. to 900° C. (specified in the Table 1). The mean residence time of the silica in the rotary kiln was 1 hour. Rotational speed was set to 5 rpm resulting in a throughput of approximately 1 kg/h of silica. Dry and filtered compressed air was fed continuously with a flow rate of ca. 1 m³/h to the kiln outlet (in counterflow to the thermally treated silica flow) to provide preconditioned air for the convection in the tube. The process was smooth. No clogging of the rotary kiln was observed. Physico-chemical properties of the obtained thermally treated silica are shown in Table 1 and Table 2.

### Absorption of 1-Propanol on Silica and Determination of the Extent of the Desorption (1-Propanol Retention) after 24 h [%]

1-Propanol (10.0% by weight of tested silica, ca. 1 g) was sprayed at ambient temperature on ca. 10 g of the silica samples directly after preparation and cooling under flow of dried air. After thorough mixing the silica, a sample thereof (M˜1 g) was taken, weighted (m₀), and spread in a thin layer in a petri dish. After 24 hours staying under air (relative humidity ca. 50%) at 25° C. in fume hood, the sample was weighted again (m₁). Mass difference (m₁-m₀) was attributed to desorbed/evaporated 1-propanol. The value “1-propanol retention after 24 h [%]” shown in the Table 1 below was calculated as 100%×[1−(m₀-m₁)×/(0.1×M)].

### Results

Surprisingly, it turned out that the extent (rate) of 1-propanol desorption from silica depended on the silanol group density of silica used for alcohol absorption. The results shown in Tables 1 and 2 indicate the optimum of 1-propanol retention at silanol group density of the precipitated silica of about 1.75 to 3.60 SiOH/nm² (examples 2 to 6), particularly 2.75 to 3.34 SiOH/nm² (examples 3 and 4). Interestingly, relatively high silanol group density of the untreated precipitated silica (3.73, Comparative Example 1) delivered inferior results of 1-propanol retention just as the relatively low silanol group density of the silica calcined at 900° C. (1.40, Comparative Example 7). The lowest aggregation rate of silica was observed in the range of silanol group density above 3.34 and below 3.73 SiOH/nm².

TABLE 1

| | Thermal | | | | | 1- |
|---|---|---|---|---|---|---|
| | treatment | | KF | Silanol | | Propanol |
| | temper- | | Water | density | | retention |
| | ature | BET | content | [SiOH/ | d₅₀ | after |
| Example | [° C.] | [m²/g] | [wt %] | nm²] | [μm] | 24 h [%] |
| Comparative | No | 180 | 6.92 | 3.73 | 320 | 20 |
| Example 1 | | | | | | |
| Example 2 | 120 | 182 | 5.10 | 3.60 | 330 | 56 |
| Example 3 | 300 | 187 | 4.20 | 3.34 | 480 | 67 |
| Example 4 | 400 | 189 | 2.70 | 2.75 | 479 | 68 |
| Example 5 | 500 | 191 | 2.49 | 2.07 | 484 | 53 |
| Example 6 | 700 | 172 | 0.90 | 1.75 | 512 | 44 |
| Comparative | 900 | 110 | 0.46 | 1.40 | 953 | 15 |
| Example 7 | | | | | | |

TABLE 2

| | Tamped density | Pore volume | DOA Absorption |
|---|---|---|---|
| Example | [g/L] | (2-50 nm) [mL/g] | [mL/100 g] |
| Comparative | 258 | 0.79 | 225 |
| Example 1 | | | |
| Example 2 | 254 | 0.77 | 221 |
| Example 3 | 242 | 0.76 | 210 |
| Example 4 | 240 | 0.84 | 207 |
| Example 5 | 259 | 0.77 | 202 |
| Example 6 | 261 | 0.78 | |
| Comparative | | 0.63 | |
| Example 7 | | | |

1. Dry mixture comprising:
(a) from 10% to 99% by weight of a precipitated silica having:
(a1) BET surface area of from 30 m²/g to 700 m²/g,
(a1) numerical median particle size d₅₀ determined by laser diffraction method of from 100 μm to 1000 μm,
(a3) silanol group density d_{SiOH} determined by reaction of the pre-dried silica with lithium aluminum hydride of from 1.55 to 3.65 SiOH/nm²
and
(b) from 1 wt % to 90 wt % of at least one organic compound:
(b1) selected from an alcohol, a diol, an aldehyde, a ketone, a carboxylic acid, a carboxylic ester, a nitrate ester, an amine, and/or oxygen and/or nitrogen heterocycle,
(b2) having 2 to 12 carbon atoms,
(b3) having a vapour pressure of at least 0.01 kPa at 293.15 K,
wherein the organic compound is adsorbed on the precipitated silica.

2. The mixture according to claim 1, wherein the organic compound has 3 to 10 carbon atoms.

3. The mixture according to claim 1, wherein the organic compound is an alcohol and/or a nitrate ester with 3 to 10 carbon atoms.

4. The mixture according to claim 1, wherein the organic compound is 3-nitrooxypropanol.

5. The mixture according to claim 1, wherein the organic compound is a mixture of ethylene glycol and 3-nitrooxypropanol.

6. The mixture according to claim 1, wherein the organic compound is a carboxylic acid with 3 to 10 carbon atoms.

7. The mixture according to claim 6, wherein the organic compound is selected from acetic acid, glyoxylic acid, propionic acid, pyruvic acid, 3-oxopropionic acid, butanoic acid, oxobutanoic acid, pentanoic acid, hexanoic acid, heptanoic acid, octanoic acid.

8. The mixture according to claim 1, wherein the organic compound is an aldehyde, a ketone, a carboxylic ester and/or an amine with 3 to 10 carbon atoms.

9. The mixture according to claim 1, wherein the organic compound is selected from estragole, ethyl maltol, terpineol, eucalyptol, linalool, dihydromyrcenol, benzaldehyde, camphor; benzyl acetate, methyl butyrate, ethyl butyrate, hexyl acetate, ethyl acetate, isoamyl acetate, methyl salicylate.

10. The mixture according to claim 1, wherein the precipitated silica has a numerical median particle size d₅₀ determined by laser diffraction method of from 200 μm to 600 μm.

11. The mixture according to claim 1, wherein the silica has a di-(2-ethylhexyl) adipate (DOA) adsorption of at least 150 mL DOA/100 g silica.

12. The mixture according to claim 1, wherein the silica is characterized by silanol group density d_{SiOH} determined by reaction of the pre-fried silica with lithium aluminum hydride of from 3.35 SiOH/nm² to 3.65 SiOH/nm².

13. The mixture according to claim 1, wherein the ratio of water content of the silica in % by weight, as determined by Karl-Fisher titration to BET surface area of the silica in m²/g is from 0.005 wt %*g/m² to 0.030 wt %*g/m².

14. The mixture according to claim 1, wherein the silica has tamped density of 150 g/L to 450 g/L.

15. A process for manufacturing the dry mixture according to claim 1, comprising:
step (a) of thermal treatment of a precipitated silica having a BET surface area of from 30 m²/g to 700 m²/g, a numerical median particle size d₅₀ determined by laser diffraction method of from 100 μm to 1000 μm and a silanol group density d_{SiOH} determined by reaction of the pre-dried silica with lithium aluminum hydride of more than 3.65 SiOH/nm² at a temperature of from 120° C. to 700° C., for at least 1 minute and
step (b) of adding at least one organic compound selected from selected from an alcohol, a diol, an aldehyde, a ketone, a carboxylic acid, a carboxylic esters, a nitrate ester, an amine, and/or oxygen or nitrogen heterocycle, having 2 to 12 carbon atoms and having a vapour pressure of at least 0.01 kPa at 293.15 K to the thermally treated silica prepared in step (a) and adsorbing the organic compound on the silica.

16. The process according to claim 15, wherein the temperature during the thermal treatment is from 120° C. to 450° C. and the duration of the thermal treatment in step (a) is at least 10 minutes.

17. The process according to claim 15, wherein essentially no water except for the adsorbed water contained in the precipitated silica prior to carrying out step (a) of the process, is added before, during or after carrying out step a) of the process.

18. The process according to claim 15, wherein step (a) is carried out under an atmosphere of gas selected from air and/or nitrogen with a maximal water content of 0.5 g water/L gas.

19. The process according to claim 15, wherein the thermal treatment in step (a) is carried out under movement of the silica at the motion rate of at least 1 cm/min, preferably in a rotary kiln or a fluidization bed reactor.

20. The process according to claim 15, wherein step (a) is carried out under an atmosphere of gas and the ratio of the employed total gas flow (in m³/h under standard conditions) to the employed total mass flow of the precipitated silica (in kg/h) is in the range 0.1 m³/kg to 10 m³/kg.
